(19) United States
(12) Patent Application Publication
(10) Pub. No.: US 2006/0123561 A1
(43) Pub. Date: Jun. 15, 2006

(54) Use of water structurants to provide fabric care benefits in a non-aqueous fabric treatment system

(75) Inventors: Jiping Wang, West Chester, OH (US); Kelli Alison Fleisch, Cincinnati, OH (US); Robb Richard Gardner, Cincinnati, OH (US); Arseni Valerevich Radomyselski, Loveland, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble & Company

(21) Appl. No.: 11/345,414

(22) Filed: Feb. 1, 2006

Related U.S. Application Data
(63) Continuation-in-part of application No. 10/238,293, filed on Sep. 10, 2002.
(60) Provisional application No. 60/650,395, filed on Feb. 4, 2005.
(60) Provisional application No. 60/687,079, filed on Jun. 3, 2005.

(51) Int. Cl. A01H 5/02; C11D 3/00

(52) U.S. Cl. 8/147; 8/115.51

(57) ABSTRACT

Water structurants are used in combination with a lipophilic fluid and water to deliver fabric care benefits to a fabric article in a non-aqueous fabric treatment system. Water structurants are effective in minimizing fabric damages (e.g., shrinkage, pilling, loss of shapes) from the fabric treatment process. Compositions comprising the water structurants for use in a non-aqueous fabric treating process are also disclosed. The compositions are capable of delivering the fabric care benefits to the fabric article and minimizing fabric damages.

### CROSS-REFERENCE TO RELATED APPLICATIONS

This application claims the benefit of U.S. Provisional Application Serial No. 60/650,395, filed on Feb. 4, 2005; U.S. Provisional Application Serial No. 60/687,079, filed on Jun. 3, 2005; and this application is a continuation-in-part application of U.S. patent application 10/238,293, filed on Sep. 10, 2002, which claims the benefits of U.S. Provisional Application Nos. 60/318,393, 60/318,439, 60/318/648, 60/318,381 and 60/318,396 all filed on Sep. 10, 2001.

### FIELD OF THE INVENTION

The present invention relates to the use of water structurants in combination with a lipophilic fluid and water to deliver fabric care benefits to a fabric article in a non-aqueous fabric treatment system. Water structurants are effective in minimizing fabric damages (e.g., shrinkage, pilling, loss of shapes) from the fabric treatment process. The present invention also discloses compositions comprising the water structurants. The compositions are capable of delivering the fabric care benefits to the fabric article in a non-aqueous fabric treating process such that fabric damages are minimized.

### BACKGROUND OF THE INVENTION

Cleaning applications typically involve the removal of foreign matter off surfaces. In laundry applications, this involves the removal of both hydrophobic and hydrophilic soils (food stains, blood, grass, dirt, grease, oils, etc.) off various fabrics including cotton, polyester, silk, rayon, wool and various blends of these materials. Typically, consumers have two choices for removal of soils: conventional water based cleaning and dry cleaning (i.e., non-aqueous based cleaning).

Conventional laundry cleaning is carried out with relatively large amounts of water, typically in a washing machine at the consumer's home, or in a dedicated place such as a coin-operated laundromat. Although washing machines and laundry detergents have become quite sophisticated, the conventional laundry process still exposes the fabric articles to the risks of dye transfer, shrinkage and wrinkling. Significant portions of fabric articles used by consumers are not suitable for cleaning in a conventional laundry process. Even fabric articles that are considered “washing machine safe” frequently come out of the laundry process badly wrinkled and require ironing.

The dry (or non-aqueous) cleaning process refers to a process where low or no water is used in the cleaning system; it uses various non-aqueous organic solvents, such as halocarbons, hydrocarbons, densified carbon dioxide, glycol ethers and silicones. By avoiding the use of large amount of water, the dry cleaning process minimizes the risk of damages to the fabric articles. Generally, water-sensitive fabrics are cleaned in this manner.

In dry cleaning processes, a small amount of water may be added, to improve the cleaning of hydrophilic stains and soils. It is believed that this added water may penetrate into the textile, causing fiber swelling, fiber movements and/or reduction in abrasion resistance such that the treated garments or fabrics exhibit unsatisfactory results including shrinkage, color fading, dye transfer, wrinkling, poor abrasion resistance, and loss of shape. These unsatisfactory results are more prominently exhibited in garments and fabrics made from hydrophilic (i.e., water sensitive) textile fibers including natural fibers, such as cotton, wool, silk, linen; regenerated cellulosic fibers, such as rayon, lyocell, and the like; and regenerated protein fibers, such as soybean fibers.

Based on the foregoing, it is desirable to have an improved non-aqueous cleaning process for treating garments or fabrics comprised of hydrophilic fibers to achieve maximal fabric cleaning or fabric care benefits (especially regarding removal of hydrophilic soils) and minimal fabric damages.

It is also desirable to have compositions for use in a non-aqueous fabric treating process that are capable of delivering the fabric care benefits to the fabric articles and minimizing fabric damages It is further desirable that the compositions contain a small amount of water and a water structurant to reduce the water penetration into the fabrics.

It is additionally desirable, though not required, that water is substantially evenly dispersed, in the droplet form, in the dry cleaning solvent matrix and the fabric treating actives are preferentially partitioned into the water droplets such that these actives are substantially uniformly deposited onto the fabric.

### SUMMARY OF THE INVENTION

The present invention relates to a fabric treating method that provides maximized fabric cleaning or fabric care benefits and minimized fabric damages to the treated fabric articles. The method comprising the steps of:
(a) obtaining a fabric treatment composition by mixing: a lipophilic fluid, a first composition comprising water and a water structurant; optionally, an emulsifying agent capable of suspending water in lipophilic fluid; and optionally, a fabric treating active;
(b) contacting a fabric article with the fabric treatment composition; and
(c) removing at least a portion of the lipophilic fluid.

The present invention also relates to a composition capable of delivering the fabric care benefits to the fabric article and minimizing fabric damages. The composition comprises at least about 50% by weight of the system of a lipophilic fluid; water; a water structurant; optionally, a fabric treating active; and optionally, an emulsifying agent capable of suspending water in lipophilic fluid such that water is in the form of discrete particles in the fabric treatment composition; wherein -weight ratio of water to water structurant ranges from about 100:1 to about 2:1.

### DETAILED DESCRIPTION OF THE INVENTION

The term “fabric article” used herein is intended to mean any article that is customarily cleaned in a conventional laundry process or in a dry cleaning process. As such the term encompasses articles of clothing, linen, drapery, and clothing accessories. The term also encompasses other items made in whole or in part of fabric, such as carpets, tote bags, furniture covers, tarpaulins, car interior, and the like.

The terms “fabric treatment composition” or “fabric treating composition” as used herein mean a dry cleaning solvent-containing composition that comes into direct contact with fabric articles to be cleaned. It is understood that the composition may also provide uses other than cleaning, such as conditioning, sizing, and other fabric care treatments. Thus, it may be used interchangeably with the term “fabric care composition”.

The term “dry cleaning” or “non-aqueous cleaning” as used herein means a non-aqueous fluid (fro example, a lipophilic fluid) is used as the dry cleaning solvent to clean a fabric article. However, water can be added to the “dry cleaning” method as an adjunct cleaning agent. The amount of water can comprise up to about 30% by weight of the dry cleaning solvent or the cleaning composition in a “dry cleaning” process.

The terms “fabric treating actives” or “actives” as used herein refer to the components that deliver the desired fabric cleaning or care benefits to the fabric article being treated. The fabric treating actives include detersive or cleaning agents that provide fabric cleaning benefits as well as fabric enhancers that provide fabric softening, odor, fabric repairs and/or improvements, and the like. On the other hand, the terms “adjunct ingredients” or “adjuncts” refer to the adjunct components incorporated into the fabric treatment composition to provide additional fabric cleaning or care benefits. The actives and adjuncts can be a liquid or a solid.

The term “soil” means any undesirable substance on a fabric article that is targeted for removal. The term “water-based soil” or “hydrophilic soil” refers to soil that comprised water at the time it first came in contact with the fabric article, that the soil has high water solubility or affinity, or the soil retains a significant portion of water on the fabric article. Examples of water-based soils include, but are not limited to beverages, many food soils, water soluble dyes, bodily fluids such as sweat, urine or blood, outdoor soils such as grass stains and mud.

The term “water soluble” as used herein means at least about 90% by weight of the fabric treating active dissolves in water, when evaluated in a 1 wt % aqueous solution. The term “water insoluble” as used herein means no more than about 10% by weight of the fabric treating active dissolves in water, when evaluated in a 1 wt % aqueous solution. The term “partially water soluble” as used herein encompasses all other fabric treating actives.

All molecular weights are weight-average molecular weights which are determined by Gel Permeation Chromatography (GPC).

Fabric Treatment Composition

The fabric treatment composition of the present invention comprises a lipophilic fluid; a first composition comprising water and a water structurant; optionally, an effective amount of an emulsifier capable of suspending water in lipophilic fluids may also be included such that water forms discrete droplets in the composition; and optionally, the fabric treatment composition may further comprise fabric treating actives and other adjunct ingredients (such as detersive surfactants, bleaches, perfumes, fabric softeners, and the like.

In one embodiment, the lipophilic fluid is selected from the group consisting of linear or cyclic silicones, glycol ethers, glycerol ethers, fluorocarbons, hydrocarbons, and mixtures thereof. In another embodiment, the lipophilic fluid comprises decamethylcyclopentasiloxane and/or other cyclic siloxane solvents. Typically, the lipophilic fluid comprises at least about 50%, or from about 60 to about 99.99%, or from about 70 to about 95%, or from about 80 to about 90%, by weight of the composition.

It is desirable to use limited amount of water in the non-aqueous fabric treating process to enhance the cleaning benefit as well as to facilitate the uniform deposition of fabric treating actives onto the fabrics. Typically, water comprises from about 0.01% to about 30% by weight of the composition. In some embodiments, water comprises less than about 10%, or less than about 5% or less than about 2% by weight of the composition. In other embodiments, water comprises at least about 0.01%, or at least about 0.1%, or at least about 0.5% by weight of the composition. Preferably, the fabric treatment composition contains discrete water droplets, which have a median particle diameter χ₅₀ of less than about 1000 μm, or less than about 500 μm, or less than about 100 μm. The median particle size is determined by the test method ISO 13320-1:1999(E), wherein χ₅₀ defined as “median particle diameter, μm” on a volumetric basis, i.e., 50% by volume of the particles is smaller than this diameter and 50% is larger. In some embodiments, the median particle size of the water droplet ranges from about 0.1 to about 1000 μm, or from about 1 to about 500 μm, or from about 5 to about 100 μm.

Water structurants are useful in the composition to minimize water penetration into the fabrics. In one embodiment, the weight ratio of water to water structurant ranges from about 100:1 to about 2:1. In other embodiments, the weight ratio of water to water structurant ranges from about 50:1 to about 3:1 or from about 40:1 to about 4:1.

In the fabric treatment composition of the present invention, a fabric treating active may be partitioned between water and the lipophilic fluid. Fabric treating actives suitable for use in the present invention may have a higher affinity for water than for the lipophilic fluid. The affinity is defined by logP, a partition coefficient of lipophilic fluid/water. In one embodiment, the fabric treating active is more soluble in water than in the lipophilic fluid. In other words, the fabric treating active has a logP of less than about 0. In another embodiment, the fabric treating active is about equally soluble in water as in the lipophilic fluid. In other words, the fabric treating active has a logP of from about −1 to about 1. A method for determining the partition coefficient of a compound in two incompatible liquids is described in “Determination of n-Octanol/Water Partition Coefficient (Kow) of Pesticides Critical Review and Comparison of Methods”, A. Finizio; M. Vighi; and D. Sandroni, Chemosphere Vol. 34(1), pages 131-161 (1997). The value of logP of a fabric treating active can be determined by adapting this partitioning method by mixing the fabric treating active with a lipophilic fluid and water. In some embodiments, the partition of fabric treating active between lipophilic fluid versus water ranges from about 1:2 to about 1:1000 (w:w).

The weight ration of water to fabric treating actives in the fabric treatment composition of the present invention ranges from about 1000:1 to about 1:3, or from about 500:1 to about 1:1, or from about 100:1 to about 3:1.

Nonlimiting examples of emulsifying agents suitable for use herein are described in details below. Suitable emulsifying agents may have a lipophilic portion and a hydrophilic portion, such as those described in US 20050003981A1 and US 20050000030A1. In a typical embodiment of the composition of the present invention, water and the emulsifying agent exhibit a weight ratio of from about 10000:1 to about 1:1, or from about 5000:1 to about 10:1, or from about 1000:1 to about 50:1. It is also known that these emulsifying agents may also function as detersive surfactants in the composition. Thus, additional amount of these emulsifying agents can also be included in the fabric treatment composition. In some embodiments, the total amount of the emulsifying agent in the fabric treatment composition to the amount of lipophilic fluid range from about 10000:1 to about 1:1 (w:w), or from about 5000:1 to about 10:1 (w:w), or from about 1000:1 to about 50:1 (w:w).

In some embodiments, co-solvents are also included in the compositions. Nonlimitng examples of co-solvents include C1-C6 linear or branched alcohols. Typically, the co-solvents comprises from about 0.01% to about 5%, or from about 0.05% to about 2%, or from about 0.1% to about 1% by weight of the composition.

(1) Water Structurants

When a fabric article is contacted by water in a laundering process, water can penetrate into the textile fibers which may result in fiber swelling and/or fiber movement. It is believed that these are contributing factors for unsatisfactory fabric treating results. It is also believed that fiber swelling and movement can result in poorer abrasion resistance and higher friction between fibers, both of which lead to fiber damages. Further, it is believed that various fabric care issues are related to these changes in fiber characteristics; specific fabric care issues include but are not limited to shrinkage (including relaxation shrinkage), wool felting, color fading, dye transfer, wrinkles.

In the present invention, the water-related damages or issues are minimized by treating the fabrics in a substantially non-aqueous process, which uses a small amount of water and water structurants in a lipophilic fluid medium. Without being bound by theory, it is believed that water structurants are capable of holding water on the fabric surface, resulting in significantly reduction of water penetration into the fabrics, thereby reducing or even eliminating fiber swelling and/or fiber movements.

Nonlimiting examples of water structurants suitable for use in the present invention include the following:
1. Inorganic and organic salts

Inorganic and organic salts composed of cationic metal ions, organic cations and various anions. The cationic metal ions include, but are not limited to, Na⁺, K⁺, Li⁺, Mg⁺⁺, Ca⁺⁺, Zn⁺⁺, La⁺⁺, La⁺⁺⁺, Al⁺⁺⁺, and the like. The organic cations include, but are not limited to, NH₄⁺, protonated primary, secondary or tertiary amines or polyamines, and the like. The anions include, but are not limited to F⁻, Cl⁻, Br⁻, I⁻, ClO₄⁻, IO₄⁻, SO₃²⁻, SO₄²⁻, PO₃³⁻, PO₄³⁻, CO₃²⁻, alkyl carboxylates (e.g., acetate, succinate, and the like) and citrate. It is desirable that the salts do not change the pH of the composition Non-limiting examples include Na₂SO₄, K₂SO₄, Li₂SO₄, MgSO₄, CaSO₄, La₂(SO₄)₃, NaCl, KCl, LiCl, MgCl₂, CaCl₂, NaBr, KBr, LiBr, LaCl₃, AlCl₃, AlBr₃, LaBr₃, and Na₃PO₄.
2. Saccharides and derivatives

Suitable monosaccharides, oligosaccharides, polysaccharides and their derivatives have a weight-average molecular weight below about 10,000 daltons and have the ability to hold water/moisture. Monosaccharides suitable for use herein include, but are not limited to, mannose, galactose, arabinose, xylose, ribose, apiose, rhamnose, psicose, fructose, sorbose, tagitose, ribulose, xylulose, glucose, and erythrulose. Suitable oligosaccharides include, but are not limited to, maltose, kojibiose, nigerose, cellobiose, lactose, melibiose, gentiobiose, turanose, rutinose, trehalose, sucrose and raffinose. Suitable polysaccharides include, but are not limited to, amylose, glycogen, cellulose, chitin, inulin, agarose, xylans, mannan and galactans; hydrolyzed form of these polysaccharides having a weight-average molecular weight below about 10,000 daltons, or below about 5,000 daltons, or below about 2000 daltons, are also suitable. Also suitable for use herein are sugar alcohols, including but are not limited to, sorbitol, erythritol, arabitol, xylitol, threitol, pentaerythritol, mannitol and galactitol.
3. Polyalkylene glycols and polyalylene polyols

Suitable polyalkylene glycols and polyalkylene polyols have a weight-average molecular weight below about 20,000 daltons. The polyalkyl glycols include polymers and copolymers derived from C2-C6 alkylene oxide monomers, such as, polyethylene glycol (PEG), polypropylene glycol (PPG), polyethylene/propylene glycol, polybutylene glycol (PBG), and the like. The polyalkylene polyols include polymers and copolymers derived from C3-C12 alkylene diol, triol or polyol monomers.
4. Hydrolyzed proteins and their derivatives with a weight-average molecular weight below about 50,000 daltons.
5. Polar nitrous organic materials

Polar nitrous organic materials, such as urea, amine, amide and their derivatives, are suitable for use herein as water structurants. Exemplary polar nitrous organic materials include urea, primary, secondary or tertiary amines or polyamines, acetamide, N-methylacetamide, dimethylformamide, guanidine, alkyl guanidinium (such as N-methyl guanidinium), and derivatives and mixtures thereof.
6. Hydrophilic polymers

Suitable hydrophilic polymers are capable of holding water. The water holding capacity of these hydrophilic polymer is at least about 100%, or about 100% to about 1000%, or about 200% to about 700%, of the weight of the polymers. Non-limiting examples include polymers and copolymers derived from monomeric units such as acrylates, methacrylates, acrylamides, acrylonitrile, vinyl alcohol, vinyl alcohol-vinyl acetate, and their derivatives. These hydrophilic polymers may be non-crosslinked or lightly (i.e., less than 10% of the monomeric units) crosslinked. Some of these hydrophilic polymers are known as the “absorbent gel materials (AGM)”.

(2) Lipophilic Fluid

Lipophilic fluid may be any liquid or mixture of liquid is immiscible with water at up to about 20% by weight of water. In general, a suitable lipophilic fluid can be fully liquid at ambient temperature and pressure, can be an easily melted solid, e.g., one that becomes liquid at temperatures in the range from about 0° C. to about 60° C., or can comprise a mixture of liquid and vapor phases at ambient temperatures and pressures, e.g., at 25° C. and 1 atm. pressure.

It is preferred that the lipophilic fluid herein be non-flammable or, have relatively high flash points and/or low VOC characteristics, these terms having conventional meanings as used in the dry cleaning industry, to equal to or exceed the characteristics of known conventional dry cleaning fluids.

Non-limiting examples of suitable lipophilic fluid materials include siloxanes, other silicones, hydrocarbons, glycol ethers, glycerine derivatives such as glycerine ethers, perfluorinated amines such as perfluorotributylamines, perfluorinated and hydrofluoroether solvents, low-volatility nonfluorinated organic solvents, other environmentally-friendly solvents and mixtures thereof.

“Siloxane” as used herein means silicone fluids that are non-polar and-insoluble in water or lower alcohols. Linear siloxanes (see for example U.S. Pat. Nos. 5,443,747, and 5,977,040) and cyclic siloxanes are useful herein, including the cyclic siloxanes selected from the group consisting of octamethyl-cyclotetrasiloxane (tetramer), dodecamethyl-cyclohexasiloxane (hexamer), decamethyl-cyclopentasiloxane (pentamer, commonly referred to as “D5”), and mixtures thereof. A preferred siloxane comprises more than about 50% cyclic siloxane pentamer, or more than about 75% cyclic siloxane pentamer, or at least about 90% of the cyclic siloxane pentamer. Also preferred for use herein are siloxanes that are a mixture of cyclic siloxanes having at least about 90% (or at least about 95%) pentamer and less than about 10% (or less than about 5%) tetramer and/or hexamer.

Other suitable lipophilic fluids include, but are not limited to, diol solvent systems e.g., higher diols such as C₆ or C₈ or higher diols, organosilicone solvents including both cyclic and acyclic types, and the like, and mixtures thereof.

Non-limiting examples of low volatility non-fluorinated organic solvents include for example OLEAN® and other polyol esters, or certain relatively nonvolatile biodegradable mid-chain branched petroleum fractions.

Non-limiting examples of glycol ethers include propylene glycol methyl ether, propylene glycol n-propyl ether, propylene glycol t-butyl ether, propylene glycol n-butyl ether, dipropylene glycol methyl ether, dipropylene glycol n-propyl ether, dipropylene glycol t-butyl ether, dipropylene glycol n-butyl ether, tripropylene glycol methyl ether, tripropylene glycol n-propyl ether, tripropylene glycol t-butyl ether, tripropylene glycol n-butyl ether.

Non-limiting examples of other silicone solvents, in addition to the siloxanes, are well known in the literature, see, for example, Kirk Othmer's Encyclopedia of Chemical Technology, and are available from a number of commercial sources, including GE Silicones, Toshiba Silicone, Bayer, and Dow Corning. For example, one suitable silicone solvent is SF-1528 available from GE Silicones.

Non-limiting examples of suitable glycerine derivative solvents for use in the present invention have the following structure: 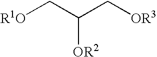
wherein R¹, R² and R³ are each independently selected from: H; branched or linear, substituted or unsubstituted C₁-C₃₀ alkyl, C₂-C₃₀ alkenyl, C₁-C₃₀ alkoxycarbonyl, C₃-C₃₀ alkyleneoxyalkyl, C₁-C₃₀ acyloxy, C₇-C₃₀ alkylenearyl; C₄-C₃₀ cycloalkyl; C₆-C₃₀ aryl; and mixtures thereof. Two or more of R¹, R² and R³ together can form a C₃-C₈ aromatic or non-aromatic, heterocyclic or non-heterocyclic ring.

Non-limiting examples of suitable glycerine derivative solvents include 2,3-bis(1,1-dimethylethoxy)-1-propanol; 2,3-dimethoxy-1-propanol; 3-methoxy-2-cyclopentoxy-1-propanol; 3-methoxy-1-cyclopentoxy-2-propanol; carbonic acid (2-hydroxy-1-methoxymethyl)ethyl ester methyl ester; glycerol carbonate and mixtures thereof.

Non-limiting examples of other environmentally-friendly solvents include lipophilic fluids that have an ozone formation potential of from about 0 to about 0.31, lipophilic fluids that have a vapor pressure of from about 0 to about 0.1 mm Hg, and/or lipophilic fluids that have a vapor pressure of greater than 0.1 mm Hg, but have an ozone formation potential of from about 0 to about 0.31. Non-limiting examples of such lipophilic fluids that have not previously been described above include carbonate solvents (i.e., methyl carbonates, ethyl carbonates, ethylene carbonates, propylene carbonates, glycerine carbonates) and/or succinate solvents (i.e., dimethyl succinates).

“Ozone Reactivity” as used herein is a measure of a VOC's ability to form ozone in the atmosphere. It is measured as grams of ozone formed per gram of volatile organics. A methodology to determine ozone reactivity is discussed further in W. P. L. Carter, “Development of Ozone Reactivity Scales of Volatile Organic Compounds”, Journal of the Air & Waste Management Association, Vol. 44, Page 881-899, 1994. “Vapor Pressure” as used can be measured by techniques defined in Method 310 of the California Air Resources Board.

In one embodiment, the lipophilic fluid comprises more than 50% by weight of the lipophilic fluid of cyclopentasiloxanes, (“D5”) and/or linear analogs having approximately similar volatility, and optionally complemented by other silicone solvents.

(3) Fabric Treating Actives

Suitable fabric treating actives can be water soluble or partially water soluble materials (e.g., bleaches, enzymes), or water insoluble liquids (e.g., perfumes). Suitable fabric treating actives also include water insoluble solids (e.g., fluoro or silicone soil release polymers).

In some embodiments of the present invention, the fabric treatment compositions comprise one or more of the following fabric treating actives: soil release polymers, bleaches, perfumes, softening agents, finishing agents, and mixtures thereof.

(a) Soil Release Polymer

The term “soil-release” as used herein refers to the ability of the fabric article to be washed or otherwise treated to remove soils that have come into contact with the fabric article. The present invention does not wholly prevent the attachment of soil to the fabric article, but hinders such attachment and improves the cleaning of the fabric article. Nonlimiting examples of soil release polymers suitable for use herein include fluorine-containing soil release polymers (fluoro-SRPs) and silicone-containing soil release polymers (Si-SRPs).

Examples of fluoro-SRPs useful in the present invention include polymers derived from perfluoroalkyl monomers, or from a mixture of perfluoroalkyl monomers and alkyl methacrylate monomers. In one embodiment, the fluoroalkyl monomer is at least about 70% by weight of the copolymer. In another embodiment, the alkyl methacrylate is stearyl methacrylate. In yet another embodiment, The alkyl methacrylate constitutes 5-25 weight % of the copolymer. The fluoro-methacrylate SRPs are disclosed in U.S. Pat. No. 6,451,717.

Exemplary fluoro-SRPs are commercially available under the trade name Repearl F35® in an aqueous suspension form from Mitsubishi, and under the trade names Zonyl TA-N®, Zonyl 7060®, Zonyl 8300®, and Zonyl 8787® from DuPont. Other suitable fluoro-SRPs are disclosed in WO 01/98384, WO 01/81285; JP 10-182814; JP 2000-273067; WO 98/4160213, and WO 99/69126.

Exemplary Si-SRPs are commercially available as DF104, DF1040, SM2125, SM2245, SM2101, SM2059 from General Electric, and 75SF Emulsion from Dow Corning. Also suitable for use herein are Si-SPRs disclosed in US 20050000028A1. Suitable Si-SRPs have a weight-average molecular weight in the range from about 1000 to about 10,000,000 daltons, or from about 5000 to about 5,000,000 daltons, or from about 10,000 to about 1,000,000 daltons. For example, when the Si-SRP is a curable aminosilicone, it tends to have a low molecular weight from about 1000 to about 100,000 daltons. The curable Si-SRP is-relatively flowable when applied to the fabrics and can be cured to form a soil repellent, film-like layer over the fabric surface. In other examples, Si-SRPs having molecular weight higher than 100,000 daltons are used in the fabric treatment composition of the present invention to deposit the Si-SRPs onto fabric surface without further curing.

Also suitable for use as soil release polymer in the present invention are water soluble modified celluloses which include, but are not limited to: carboxymethylcellulose, hydroxypropylcellulose, methylcellulose, and like compounds. These compounds, and other suitable compounds, are described in Kirk Othmer Encyclopedia of Chemical Technology, 4^{th} Edition, vol. 5, pages 541-563, under the heading of “Cellulose Ethers”, and in the references cited therein.

Another class of suitable soil release polymers may comprise block copolymers of polyalkylene terephthalate and polyoxyethylene terephthalate, and block copolymers of polyalkylene terephthalate and polyethylene glycol. These compounds are disclosed in details in are discussed in U.S. Pat. No. 6,358,914 and U.S. Pat. No. 4,976,879.

Another class of soil release polymer is a crystallizable polyester comprising ethylene terephthalate monomers, oxyethylene terephthalate monomers, or mixtures thereof. Examples of this polymer are commercially available as Zelcon 4780® (from DuPont) and Milease T® (from ICI). A more complete disclosure of these soil release agents is contained in EP 0 185 427.

(b) Bleach

Nonlimiting examples of suitable bleaches or bleach systems are selected from the group consisting of catalytic metal complexes, activated peroxygen sources, bleach activators, bleach boosters, photobleaches, free radical initiators and hyohalite bleaches.

Examples of suitable catalytic metal complexes include, but are not limited to, manganese-based catalysts such as those disclosed in U.S. Pat. No. 5,576,282; cobalt based catalysts such as those disclosed in U.S. Pat. No. 5,597,936; and transition metal complexes of a macropolycyclic rigid ligand—abbreviated as “MRL”, such as those disclosed in WO 00/332601, and U.S. Pat. No. 6,225,464. Non-limiting examples of suitable metals in the MRLs include Mn, Fe, Co, Ni, Cu, Cr, V, Mo, W, Pd, and Ru in their various oxidation states. Non-limiting examples of suitable MRLs include dichloro-5,12-diethyl-1,5,8,12-tetraazabicyclo[6.6.2]hexadecane manganese(II), dichloro-5,12-diethyl-1,5,8,12-tetraazabicyclo[6.6.2]hexadecane manganese(III) hexafluorophosphate and dichloro-5-n-butyl-12-methyl-1,5,8,12-tetraaza-bicyclo[6.6.2] hexadecane manganese(II).

Suitable activated peroxygen sources include, but are not limited to, preformed peracids, a hydrogen peroxide source in combination with a bleach activator, or a mixture thereof. Suitable preformed peracids include, but are not limited to, compounds selected from the group consisting of percarboxylic acids and salts, percarbonic acids and salts, perimidic acids and salts, peroxymonosulfuric acids and salts, and mixtures thereof. Suitable sources of hydrogen peroxide include, but are not limited to, compounds selected from the group consisting of perborate compounds, percarbonate compounds, perphosphate compounds and mixtures thereof. Suitable types and levels of activated peroxygen sources are found in U.S. Pat. Nos. 5,576,282, 6,306,812 and 6,326,348.

Suitable bleach activators include, but are not limited to, perhydrolyzable esters and perhydrolyzable imides, tetraacetyl ethylene diamine, octanoylcaprolactam, benzoyloxybenzenesulphonate, nonanoyloxybenzenesulphonate, benzoylvalerolactam, dodecanoyloxybenzenesulphonate.

Suitable bleach boosters include, but are not limited to, those described U.S. Pat. No. 5,817,614.

(c) Perfume and Perfume Delivery System

As used herein the term “perfume” is used to indicate any odoriferous material. Suitable perfumes include but are not limited to one or more aromatic chemicals, naturally derived oils and mixtures thereof. Chemical classes for such aromatic chemicals and essential oils include but are not limited to alcohols, aldehydes, esters, ketones. Perfume is commonly provided with a perfume delivery system.

Suitable perfume delivery systems include but are not limited to perfume loaded cyclodextrins, amine assisted delivery systems, polymer-assisted perfume systems, reactive/pro-perfume systems and inorganic carrier systems. Perfume loaded cyclodextrin delivery systems comprise perfume materials or blends complexed with cyclodextrin type materials—a majority of the cyclodextrin may be alpha-, beta-, and/or gamma-cyclodextrin, or simply beta-cyclodextrin. Processes for producing cyclodextrins and cyclodextrin delivery systems are further described in U.S. Pat. Nos. 3,812,011, 4,317,881, 4,418,144 and 5,552,378.

Amine assisted delivery systems comprise one or more perfumes and a polymeric and/or non-polymeric amine material that is added separately from the perfume to the finished products. Such systems are described in WO 03/33635 and WO 03/33636.

Polymer-assisted delivery systems use physical bonding of polymeric materials and perfumes to deliver perfume materials. Suitable polymer assisted systems, include but not limited to, reservoir systems (coacervates, microcapsules, starch encapsulates), and matrix systems (polymer emulsions, latexes). Such systems are further described in WO 01/79303, WO 00/68352, WO 98/28339, and U.S. Pat. Nos. 5,188,753 and 4,746,455.

Reactive/pro perfumes systems include, but are not limited to, polymeric pro-perfumes that comprise perfume materials, typically aldehyde or ketone perfumes, reacted with polymeric carriers, typically nitrogen based carriers, prior to addition to a product; non-polymeric pro-perfume systems that comprise perfume materials reacted with non-polymeric materials for example, Michael adducts (β-amino ketones), Schiff bases (imines), oxazolidines, β-keto esters, orthoesters and photo pro-perfumes. Such systems are further described in WO 00/24721, WO 02/83620 and U.S. Pat. Nos. 6,013,618 and 6,451,751.

Inorganic carrier systems that comprise inorganic materials (porous zeolites, silicas, etc.) that are loaded with one or more perfume materials. Such systems are further described in U.S. Pat. Nos. 5,955,419, 6,048,830 and 6,245,732.

(d) Softening Agents

Suitable fabric softening agents or actives include, but are not limited to, diester quaternary ammonium compounds (DEQA); polyquaternary ammonium compounds; triethanolamine esterified with carboxylic acid and quaternized (so called “esterquat”); amino esterquats; cationic diesters; betaine esters; cyclic polyols and/or reduced saccharides and cationic polymers derived thereof (for example, polyol polyesters including sucrose ester, and hydrolyzed cationic starch); silicone or silicone emulsions comprising aminosilicones, cationic silicones, quat/silicone mixtures; functionalized PDMS; and mixtures thereof.

Deposition aids, typically comprise a cationic moiety, can also be used in combination with softening agents.

Nonlimiting examples of quaternary ammonium type softeners may be selected from the group consisting of: N,N-dimethyl-N,N-di(tallowyloxyethyl) ammonium methylsulfate, N-methyl-N-hydroxyethyl-N,N-di(canoyloxyethyl) ammonium methylsulfate and mixtures thereof.

Additional examples of non-silicone fabric softening agents and deposition aids are described in EP 902 009; WO 99/58492; U.S. Pat. No. 4,137,180; WO 97/08284; WO 00/70004; WO 00/70005; WO 01/46361; WO 01/46363; WO 99/64661; WO 99/64660; JP 11-350349; JP11-081134; and JP 11-043863. Additional examples of silicone fabric softening agents and deposition aids are described in U.S. Pat. No. 4,448,810; U.S. Pat. No. 4,800,026; U.S. Pat. No. 4,891,166; U.S. Pat. No. 5,593,611; EP 459 821; EP 530 974; WO 92/01773; WO 97/32917; WO 00/71806; WO 00/71807; WO 01/07546; WO 01/23394; JP 2000-64180; JP 2000-144199; JP 2000-178583; and JP 2000-192075.

(e) Finishing Polymers

The finishing polymers can be natural, or synthetic, and can act by forming a film, and/or by providing adhesive properties. For example, the present invention can optionally use film-forming and/or adhesive polymer to impart shape retention to fabric, particularly clothing. By “adhesive” it is meant that when applied as a solution or a dispersion to a fiber surface and dried, the polymer can attach to the surface. The polymer can form a film on the surface, or when residing between two fibers and in contact with the two fibers, it can bond the two fibers together.

Nonlimiting examples of the finishing polymer that are commercially available are: polyvinylpyrrolidone/dimethylaminoethyl methacrylate copolymer, such as Copolymer 958® and Copolymer 937®, available from GAF Chemicals Corporation; adipic acid/dimethylaminohydroxypropyl diethylenetriamine copolymer, such as Cartaretin F-4® and F-23, available from Sandoz Chemicals Corporation; methacryloyl ethyl betaine/methacrylates copolymer, such as Diaformer Z-SM®, available from Mitsubishi Chemicals Corporation; polyvinyl alcohol copolymer resin, such as Vinex 2019®, available from Air Products and Chemicals or Moweol®, available from Clariant; adipic acid/epoxypropyl diethylenetriamine copolymer, such as Delsette 101®, available from Hercules Incorporated; polyamine resins, such as Cypro 515®, available from Cytec Industries; polyquaternary amine resins, such as Kymene 557H®, available from Hercules Incorporated; and polyvinylpyrrolidone/acrylic acid, such as Sokalan EG 310®, available from BASF.

Additional examples of suitable finishing polymers include but are not limited to starch carboxymethyl cellulose, hydroxypropyl methyl cellulose, polylysines, and mixtures thereof.

(g) Other Fabric Treating Actives

Various fabric treating actives may also be incorporated in the composition of the present invention. Nonlimiting examples of suitable fabric treating actives include enzymes, dye transfer inhibiting agents, dye fixatives, UV protection agents, wrinkle reducing/removing agents, fabric rebuild agents, fiber repair agents, perfume release and/or delivery agents, shape retention agents, fabric and/or soil targeting agents, antibacterial agents, anti-discoloring agents, UV blockers, brighteners, pigments (e.g., AL₂O₃, TiO₂), pill prevention agents, temperature control technology, skin care lotions (comprising one or more of humectants, moisturizers, viscosity modifiers, and fragrances), and fire retardants. Examples of suitable fabric treating actives are disclosed in U.S. Pat. No. 6,673,764; U.S. Pat. No. 6,746,617; U.S. Pat. No. 6,734,153; U.S. Pat. No. 6,660,703; US 20040266643A1; US 20040261195A1; US 20040261196A1; and US 20050000028A1.

(4) Emulsifying Agent

Suitable emulsifying agents or emulsifiers may comprise a lipophilic portion and a hydrophilic portion, and are capable of suspending water in lipophilic fluids. Detailed description of these emulsifiers is found in U.S. Pat. No. 20050003981A1 and U.S. Pat. No. 20050000030A1.

Nonlimiting examples of emulsifiers having the above formula include alkanolamines; phosphate/phosphonate esters; gemini surfactants including, but are not limited to, gemini diols, gemini amide alkoxylates, gemini amino alkoxylates; capped nonionic surfactants; capped silicone surfactants such as nonionic silicone ethoxylates, silicone amine derivatives; alkyl alkoxylates; polyol surfactants; and mixtures thereof.

Other suitable emulsifiers are organosulfosuccinates, with carbon chains of from about 6 to about 20 carbon atoms. In one embodiment, the organosulfosuccinates contain dialkly chains, each with carbon chains of from about 6 to about 20 carbon atoms. In another embodiment, the organosulfosuccinates have chains containing aryl or alkyl aryl, substituted or unsubstituted, branched or linear, saturated or unsaturated groups. Nonlimiting commercially available examples of suitable organosulfosuccinate surfactants are available under the trade names of Aerosol OT® and Aerosol TR-70® (ex. Cytec).

Method

The present invention also comprises a method of efficient and uniform deposition of a fabric treating active onto a fabric article in a non-aqueous solvent based fabric treatment process. The method typically comprises the steps of: obtaining the fabric treatment composition comprising a lipophilic fluid, water, one or more water structurants, optionally, one or more fabric treating actives and optionally, an emulsifying agent; applying the fabric treatment composition to a fabric article; and removing at least a portion of the lipophilic fluid from the fabric treatment composition. Optionally, lipophilic fluid and/or water in addition to the fabric treatment composition may be applied to the fabric article.

The fabric treatment composition can be applied to the fabric article by immersing, dipping, spraying, brushing on, rubbing on, and combinations thereof. The fabric treatment composition can be applied to a fabric article in a treatment apparatus during the washing cycle, the drying cycle or a fabric refreshing/treating cycle. The fabric treatment composition can also be applied to a fabric article outside of a treatment apparatus, for example, in a pre-or post-laundering step.

The fabric treatment composition can be prepared prior to being added to the treatment apparatus. Mechanical energy (such as stirring, shaking or vortexing) may be used during the preparation of the composition to help breaking up the water droplets to the desired size range and partitioning the actives between water and lipophilic fluid.

Alternatively, one or more components of the fabric treatment composition can be added to separate holding tanks or containers within the treatment apparatus and mixed in the treatment apparatus to form the fabric treatment composition prior to being applied to the fabric article. In one embodiment, the lipophilic fluid and water (including water structurants) are applied to the fabric article from different containers or sources; fabric treating actives and emulsifying agents are optionally premixed into either source.

The lipophilic fluid can be removed from the treated fabric article by heating, spinning, squeezing, wringing, or combinations thereof.

A desired amount of fabric treating active may be deposited in one cycle or the same desired amount of fabric treating active may be divided or separated into smaller amounts and the fabric treating method may be applied more that one time such that fabric treating active is deposited in small amounts over a series of cycles to cumulatively deposit the desired amount of fabric treating active on the fabric article.

An optional step of the method is the removal of the fabric treatment composition from the fabric article prior to heating of the fabric article.

Any suitable fabric article treating apparatus known to those of ordinary skill in the art can be used. The fabric article treating apparatus receives and retains a fabric article to be treated during the operation of the cleaning system. In other words, the fabric article treating apparatus retains the fabric article while the fabric article is being contacted by the fabric treating composition. Nonlimiting examples of suitable fabric article treating apparatuses include commercial dry cleaning machines, or in-home dry cleaning machines, such as those described in U.S. Pat. No. 6,691,536.

The methods and fabric treatment compositions of the present invention may be used in a service, such as a cleaning service, diaper service, uniform cleaning service, or commercial business, such as a Laundromat, dry cleaner, linen service which is part of a hotel, restaurant, convention center, airport, cruise ship, port facility, casino, or may be used in the home.

The methods of the present invention may be performed in an apparatus that is a modified existing washer/dryer and is retrofitted in such a manner as to conduct the method of the present invention in addition to related methods.

The methods of the present invention may also be performed in an apparatus that is specifically built for conducting the present invention and related methods.

Further, the methods of the present invention may be added to another apparatus as part of a dry cleaning solvent processing system. This would include all the associated plumbing, such as connection to a chemical and water supply, and sewerage for waste wash fluids.

### TEST METHOD AND EXAMPLES

The following test method is used to show that the fabric treatment composition of the present invention is capable of control shrinkage of the treated fabrics.

Care Performance Test Method:

Apparatus: Atlas LP2 Launder-Ometer (available from SDL Atlas L.L.C., Charlotte, N.C.).

Sample Swatches:

100% Wool Flannel—type #527, available from Test Fabrics Inc. West Pittston, Pa.
100% Cotton Twill—type CSBI, available from Cotton Inc. Cary, N.C.
   Swatch Preparation: take one 10″−10″ (25.4 cm×25.4 cm) swatch and mark it with an indelible pen; make three pairs of marks along each of the warp (W) and fill (F) directions, each mark is about 1″ (2.54 cm) from edge of fabric. Label each sample swatch for identification.
   Initial Measurements: Take the initial measurements of the distance between the corresponding pairs of marks in the Fill (F) direction and in the Warp (W) direction.
   Ballast (available from Test Fabric Inc.): 3½″×3½″ (8.9 cm×8.9 cm) swatches of the following: 1 piece of W541, 1 piece of S601, 3 pieces of CW120, 3 pieces of CW25 and 3 pieces of PCW 50/50 are used to accompany each testing sample in one canister.
1. Fill the water bath of the Launder-Ometer with room temperature water (about 25° C.). Fill to the “Water-Line” mark located inside the bath.
2. Obtain Type 2 Canisters (Part No. 11241600 of Launder-Ometer, available from Atlas) and label the canisters.
3. Add 50 stainless steel (SS) ball bearings (6 mm diameter, total weight of about 53.5 g) to each canister.
4. Add the appropriate amount of decamethylcyclopentasiloxane, commonly known as D5 (available as SF1202® from GE Silicones) to achieve a fluid to fabric weight ratio of 10 to 1. Thus, for Wool 527 samples, about 430 mL D5 is added to each canister; for Cotton CSB1 samples, about 500 mL D5 is added to each canister.
5. Add the detergent to the canister; the amount the detergent is relative to the amount of D5 in the canister; for the purpose of this experiment, detergent to D5 is 0.5:100 w:w.
6. Place a viton seal (available from McMaster-Carr, Aurora, Ohio, Part# 8625-K29) inside the cover; be sure to use viton or other D5 resistant seals. Seal the canister and shake it by hand for about 15 seconds to mix the detergent and D5.
7. Open the canister and add varying amounts of water or water/structurant mixture (see Examples below) to the canister. Seal the canister and shake it vigorously by hand for about 30 seconds to achieve thorough mixing of the components.
8. Open the canister; randomly pick out about half of the ballast swatches and add them to the canister, then loosely roll the sample swatch (with the marked portion of the fabric on the outside) and add it to the canister; finally, add the remaining ballast swatches to the canister.
9. Reseal the canister. Place canisters in the Launder-Ometer. If an odd number of canisters are used, balance the load with a dummy canister of approximately the same weight.
10. After all canisters are in place, close the lid. Run the Launder-Ometer on Program PT No.1. Start the program by pressing the [symbol] key for >3 seconds. Set a timer for 60 min. as the program must be stopped manually. After 60 min. press the [symbol] key for >5 seconds to stop the program.
11. After the rotor has come to a stop, remove the canisters from Launder-Ometer.
12. Open the canisters and take out all the ballast and sample swatches. Gently squeeze the sample swatches to remove the excess liquid.
13. Lay the sample swatch out flat on a fabric support, which is a piece of cotton fabric having a dimension larger than the sample swatch. Attach the four corners of the sample swatch to the fabric support using a tagging gun or equivalent means. Attach one sample swatch per fabric support.
14. Tumble dry the sample/support ensembles on “cotton high” setting in a Kenmore (available from Sears) compact dryer for about 60 min or until dry. Equivalent dryers set at equivalent temperature can be used instead to dry the sample swatches.
   Shrinkage Measurements:
1. After the dried sample/support ensemble has cooled to room temperature, lay the sample swatch on a flat surface (remove the support if necessary) and take the Final Measurements of the distance between the corresponding pairs of marks that was measured initially in the Fill (F) and in the Warp (W) directions.
2. Use the following calculation to obtain a % Dimensional Change for each measurement:

(a) Shrinkage in Warp Direction (S_{w}):
S_{w}=100×(B_{w}−A_{w})/A_{w}
wherein B_{w} is the average Final measurement in warp direction and A_{w} is the average Initial measurement in warp direction; A_{w} and B_{w} are averaged over three pairs of marks;

(b) Shrinkage in Fill Direction (S_{f}):
S_{f}=100×(B_{f}−A_{f})/A_{f}
wherein B_{f} is the average Final measurement in fill direction and A_{f} is the average Initial dimension in fill direction; A_{f} and B_{f} are averaged over three pairs of marks;

(c) Area Shrinkage (AS):
AS=100×(B_{w}B_{f}−A_{w}A_{f})/A_{w}A_{f}

At least two and preferably three sample swatches are tested. The dimensional changes (S_{w}, S_{f} and AS) from the sample swatches are averaged and reported. If the % change is negative, it is shrinkage and if it is positive, it is stretch.

### EXAMPLES

Amounts of detergent, water and water structurant are relative to amount of D5, which is set to 100 wt %. Tergitol® is available from Dow Chemicals; TSF® and Silwet® are available from GE silicones.

| | I | II | III | IV | V |
|---|---|---|---|---|---|
| Detergent* | 0.5% | 0.5% | 0.5% | 0.5% | 0.5% |
| Water | 1% | 1% | 1% | 1% | 1% |
| Na₂SO₄ | None | 0.25% | None | None | 0.15% |
| MgSO₄ | None | None | 0.10% | 0.30% | 0.10% |
| Shrinkage | | | | | |
| Wool Flannel: | | | | | |
| Warp | −13.5% | −4.7% | −6.6% | −2.6% | −4.7% |
| Fill | −9.2% | −3.3% | −3.7% | −2.0% | −2.6% |
| Area | −21.5% | −7.8% | −10.1% | −4.5% | −7.2% |
| Cotton Twill: | | | | | |
| Warp | −1.9% | −1.0% | −1.1% | −0.7% | −0.8% |
| Fill | −7.6% | −5.0% | −4.9% | −3.2% | −4.8% |
| Area | −9.3% | −6.0% | −5.9% | −3.9% | −5.6% |

*detergent composition used for this test contains Tergitol 15-S-3 ® (50%), TSF4446 ® (20%) and 1,2-hexanediol (30%); amounts are based on weight percent of the composition.

| | VI | VII |
|---|---|---|
| Detergent* | 0.5% | 0.5% |
| Water | 1% | 1% |
| PEG1000 | None | 0.25% |
| Shrinkage | | |
| Wool Flannel: | | |
| Warp | −16.3% | −3.7% |
| Fill | −11.7% | −2.4% |
| Area | −26.1% | −6.0% |
| Cotton Twill: | | |
| Warp | −8.6% | −7.4% |
| Fill | −2.4% | −1.6% |
| Area | −10.8% | −8.9% |

*detergent composition used for this test contains Tergitol 15-S-3 ® (46.7%), TSF4446 ® (10%), Silwet 7280 ® (13.7%) and 1,2-hexanediol (29.6%).

Examples I and VI are the control where the fabric treating composition contains no water structurant; Example II-V and VI are embodiments of the compositions of the present invention. Reduction in shrinkage is observed when water structurants are used in the fabric treating compositions.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All percentages stated herein are by weight unless otherwise specified. It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited are, in relevant part, incorporated herein by reference; the citation of any document is not to be construed as an admission that it is prior art with respect to the present invention.

What is claimed is:

1. A method for treating a fabric article in a non-aqueous fabric treatment process comprising the steps of:
(a) obtaining a fabric treatment composition by mixing:
(i) a lipophilic fluid;
(ii) a first composition comprising water and a water structurant;
(iii) optionally, an emulsifying agent capable of suspending water in lipophilic fluid; and
(iv) optionally, a fabric treating active;
(b) contacting a fabric article with the fabric treatment composition; and
(c) removing at least a portion of the lipophilic fluid.

2. The method of claim 1 wherein the water structurant is selected from the group consisting of: inorganic and organic salts; polyalkylene glycols and polyalkylene polyols; saccharides or derivatives; hydrolyzed proteins; polar nitrous organic materials; hydrophilic polymers capable of holding an amount of water at least about 100% by weight of the polymer; and mixtures thereof.

3. The method of claim 1 wherein weight ratio of water to water structurant ranges from about 100:1 to about 2:1.

4. The method of claim 1 wherein water comprises from about 0.01% to about 30% by weight of the fabric treatment composition.

5. The method of claim I wherein the lipophilic fluid comprises at least about 50% by weight of the fabric treatment composition.

6. The method of claim 1 wherein the lipophilic fluid is a cyclic siloxane selected from octamethyl-cyclotetrasiloxane, decamethyl-cyclopentasiloxane, dodecamethyl-cyclohexasiloxane, and mixtures thereof.

7. The method of claim 1 wherein the fabric treatment composition further comprises a co-solvent of linear or branched C1-C6 alcohols.

8. The method of claim 1 wherein the fabric treating active is selected from the group consisting of soil release polymers, bleaches, enzymes, perfumes, softening agents, finishing polymers, dye transfer inhibiting agents, dye fixatives, UV protection agents, wrinkle reducing/removing agents, fabric rebuild agents, fiber repair agents, perfume release and/or delivery agents, shape retention agents, fabric and/or soil targeting agents, antibacterial agents, anti-discoloring agents, hydrophobic finishing agents UV blockers, brighteners, pigments, pill prevention agents, temperature control technology, skin care lotions, fire retardants, and mixtures thereof.

9. The method of claim 1 wherein the fabric treating active is selected from the group consisting of soil release polymers, bleaches, perfumes, softening agents, finishing polymers, and mixtures thereof.

10. A fabric treatment composition for use in a non-aqueous fabric treatment process, the fabric treatment composition comprising:
(a) at least about 50% by weight of the system of a lipophilic fluid;
(b) water;
(c) a water structurant selected from the group consisting of inorganic and organic salts; polyalkylene glycols and polyalkylene polyols; saccharides or derivatives;
hydrolyzed proteins; polar nitrous organic materials; hydrophilic polymers capable of holding an amount of water at least about 100% by weight of the polymer; and mixtures thereof;
(d) optionally, a fabric treating active; and
(e) optionally, an emulsifying agent capable of suspending water in lipophilic fluid such that water is in the form of discrete particles in the fabric treatment composition;
wherein weight ratio of water to water structurant ranges from about 100:1 to about 2:1.

11. The fabric treatment composition of claim 10 wherein a weight ratio of water to the emulsifying agent ranges from about 10000:1 to about 1:1.

12. The fabric treatment composition of claim 10 wherein a weight ratio of the fabric treating active to water ranges about 1:1000 to about 3:1.

13. The fabric treatment composition of claim 10 wherein amount of fabric treating active in the lipophilic fluid versus water ranges from about 1:2 to about 1:1000.

14. The fabric treatment composition of claim 10 wherein the lipophilic fluid is a cyclic siloxane selected from octamethyl-cyclotetrasiloxane, decamethyl-cyclopentasiloxane, dodecamethyl-cyclohexasiloxane, and mixtures thereof.

15. The fabric treatment composition of claim 10 wherein water comprises from about 0.01% to about 30% by weight of the fabric treatment composition.

16. The fabric treatment composition of claim 10 wherein the fabric treatment composition further comprises a co-solvent of linear or branched C1-C6 alcohols.

17. The fabric treatment composition of claim 10 wherein the fabric treating active is selected from the group consisting of soil release polymers, bleaches, enzymes, perfumes, softening agents, finishing polymers, dye transfer inhibiting agents, dye fixatives, UV protection agents, wrinkle reducing/removing agents, fabric rebuild agents, fiber repair agents, perfume release and/or delivery agents, shape retention agents, fabric and/or soil targeting agents, antibacterial agents, anti-discoloring agents, UV blockers, brighteners, pigments, pill prevention agents, temperature control technology, skin care lotions, fire retardants, and mixtures thereof.

18. The fabric treatment composition of claim 10 wherein the fabric treating active is selected from the group consisting of soil release polymers, bleaches, perfumes, softening agents, finishing polymers, and mixtures thereof.

19. The fabric treatment composition of claim 10 wherein the fabric treating active has a logP value of less than 0 or from about 1 to about -1.

20. The fabric treatment composition of claim 10 wherein the fabric treating active is a water soluble or partially water soluble material, a water insoluble liquid, or a water insoluble solid.
